SEQUENCE LISTING (1 sequence)

SEQ ID NO: 1 - DNA, 3117 bp, Mus musculus
ccgggtcgacccacgcgtcgcgagggcagcctgggatgcgggcacggtcccggacagccccattctgccttcacgctaatgatgctggaccacaccagagcccctgagctcaacattgacctagaccttcacgcctccaactcgcctaaggggtccatgaagggcaaccaattcaaggagcaagacccttgtcctcctcagcccatgcaaggactggggaagggggacaaacgtgaagagcagggcctgggcccggaaccctcagcaccccggcagcccaccgaggaggaggaggcactgattgaattccaccgctcctaccgggagctcttccagttcttctgcaacaacaccaccatccacggggccatccgcctggtgtgctccaaacacaaccgcatgaagacggccttctgggcggtgctgtggctgtgcaccttcggcatgatgtactggcagttcgccttgctgttcgaggagtacctcagctacccagtgagcctcaacatcaacctcaattcagacaagctggtcttccctgccgtcactgtctgcacccttaatccttacagatacactgaaattaaagaggagctggaagagctggaccgcatcacggagcagacgctttttgacttgtacaaatacaactcttcctacactcgccaggctggggcccgacgccgcagctcccgcgacctcctgggtgctttcccgcaccccctgcagcgcctgcgcactccacctccgccctactccggccgcacggcgcgcagcgggtcttccagcgtacgcgacaacaatccccaagtggaccggaaggactggaagatcggcttccaactgtgcaaccagaacaaatcagactgtttctaccagacatactcctctggggtggatgcagtgagggagtggtaccgcttccattacatcaacattctgtccagactgtcggacacctcgcccgctctagaggaagaagccctgggcaacttcatcttcacctgtcgcttcaaccaggccccctgcaaccaggcgaattattccaagttccaccaccccatgtacgggaactgctacactttcaatgacaagaacaactccaatctctggatgtcctccatgcctggagtcaacaatggtttgtccctgacactgcgcacagagcagaatgacttcatccccctgctgtccacagtgacgggggccagggtgatggtgcatggtcaggatgagcctgcctttatggatgatggtggcttcaacttgaggcctggcgtggagacctccatcagtatgagaaaggaagccctggacagcctcggaggaaattacggcgactgtactgagaatggtagcgatgtcccggtcaagaacctttacccttccaagtatacacagcaggtgtgcattcactcctgcttccaggagaacatgatcaagaagtgtggctgtgcctacatcttctaccctaagcccaagggagttgagttctgtgactaccgaaagcagagctcctggggctattgctattataaactgcagggcgccttctccttggacagcctgggctgtttctccaagtgtcggaagccttgtagtgtgatcaactacaaactctctgccggctactcacggtggccatctgtgaagtcccaggattggatcttcgagatgctgtccttgcagaacaattacactattaacaacaaaagaaacggagttgcaaagctcaacatcttcttcaaggagctgaactataaaactaattcggagtctccttctgtcacgatggtcagcctcctgtccaacctgggcagccagtggagcctgtggtttggctcgtccgtgctctctgtggtggagatggcggacgtcatcttcgacctcctggtcatcacacttctcatgctgctacgccggttccggagccggtactggtctccaggacgaggggccaggggtgccagggaggtggcctccactccagcttcctccttcccgtcccgtttctgtcctcaccctacatccccaccaccttctttgccccagcagggcatgacccctcccctggccctgacagcccctccacctgcctatgctactctaggccccagtgcccctccactggactctgcggcgcctgactgttctgcctgtgccctggcggcgctctgagagaggagaaggatcctctcacccagccctgagctcccttgtaaactgatggatatctcacttcagcacatcttccccagcggccccgccgagccctctgtggtgtgcctgcctgagggaacaggctgagtaaggggcccaggaagccgtccagaggacaggggctaacgatctgctcagagctgccctgcccctgcttctgaacactgccttccacaagcacagacaagttccctcttccctcggatcagcccagccagacttggagctctgacaaggaactttcctgggagaggaccaaggaacaaatagaacagccatcgcactcctacccccgtgaccagaggctggcctggcctcactgccttcagggacactgatgtctgctcctctccttgaacttgggtggggaaccccacccaaaagcccccttgttagttctttggcaattcccctcccctcactcctcagggtggggactagagtaagacagaccagggtaggcctggtgttctctctcattctcaagactctctcatttgatacaccataccccagtgcctctgtggtgttgtctccttctttctgtttcctcagcctggaagcttctttcactgtccctcagaacacacagtctatgcatcccttagaaacctgcccaaaggcaccatttcttttgtgaactcagttaccctgttatgtctcccccagaattgtcgccctctcccctcacccccacagtgtactgtattatatgttcacattcttttgtgtctgcctccctgggtaaactgagctccttgtgtggtcagggatgagatttgctctgttttgtatccttccggtctagcccagtctcccacttgggacgggtaggcaggtactcaataaaggcttgttccatcaaaaaaaaaaaaaaaaaaaaaaaaaaaaaaaaaa

(19) United States
(12) Patent Application Publication
(10) Pub. No.: US 2003/0167494 A1
(43) Pub. Date: Sep. 4, 2003

(54) Activated cation channel of the osteoblast as a mechanism of bone anabolism

(75) Inventor: Keith Hruska, St. Louis, MO (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Barnes-Jewish Hospital

(21) Appl. No.: 10/127,276

(22) Filed: Apr. 22, 2002

Related U.S. Application Data
(63) Continuation of application No. 09/454,334, filed on Dec. 3, 1999, now abandoned.
(60) Provisional application No. 60/110,932, filed on Dec. 5, 1998.
(60) Provisional application No. 60/111,676, filed on Dec. 10, 1998.

(51) Int. Cl. A01K 67/027; C12N 5/06; C12N 15/00

(52) U.S. Cl. 800/18; 800/21; 435/320.1; 435/354

(57) ABSTRACT

A transgenic non-human animal comprising a nucleotide construct capable of altered expression of the alpha-subunit of the epithelial sodium channel which is expressed at high levels in the osteoblast lineage, and compositions, constructs, cells and methods related thereto.

This application is a continuation application of application Ser. No. 09/454,334 filed Dec. 3, 1999, which claims priority to provisional application serial No. 60/110,932 filed Dec. 5, 1998, and to provisional application serial No. 60/111,676, filed Dec. 10, 1998, the contents of all of the above applications are herein incorporated by reference.

This invention was made with Government support under National Institute of Health grant No. AR39561. The Government has certain rights in the invention.

### BACKGROUND OF THE INVENTION

The present invention relates to compositions, constructs, cells and animals with altered expression of stretch-activated cation channel, a macromolecule of the alpha-subunit of the epithelial sodium channel (α-ENaC) which is expressed at high levels in the osteoblast lineage and relates to methods of effecting bone anabolism.

It has been shown that bone formation can be stimulated by applying mechanical strain to osteocytes. The alpha-subunit of the epithelial sodium channel can form a macromolecule to produce a non-selective stretch-activated cation channel. Applying mechanical strain to these non-selective, stretch-activated cation channels located in the osteoblast plasma membrane results in an accretion of bone and an increase in skeletal/mineral density and strength. Thus, development of pharmaceutical agents which activate the stretch-activated cation channel in osteocytes and osteoblasts will serve to stimulate bone formation.

Compositions, constructs, cells and animals which allow for altered expression of stretch-activated cation channel have been needed to better understand the role of the stretch-activated cation channel in osteoblast formation. They would also be helpful in the investigation of therapeutical regimens for activating stretch-activated cation channel activity in humans and other mammals and could be used to assess various dosages of drug candidates to determine whether activation of the stretch-activated cation channel has been achieved. Another possible use for these compositions, constructs and transgenic animals is to assess the effect of another protein or combination of proteins with the stretch-activated cation channel in bone anabolism.

### SUMMARY OF THE INVENTION

Among the objects of the invention, therefore, may be noted the provision of essentially purified and/or synthetically created nucleic acid sequence which encode stretch-activated cation channel and the osteocalcin promoter, the provision of nucleic acid construct pKBpA/α-rENaC, cells and related vehicles useful in the making of animals with altered expression of stretch-activated cation channel in osteoblasts, especially mice, the provision of novel transgenic animals, such as mice, with altered expression of stretch-activated cation channel in osteoblasts, and the provision of methods of making and using such compositions, constructs and animals.

Present in the transgenic animals of the present invention are osteoblast cells which resemble those found in wild-type animals. However, the osteoblast cells in these transgenic mice have altered expression of stretch-activated cation channel due to the incorporation of the nucleotide construct pKBpA/α-rENaC into the genome of the animal. No phenotypic abnormalities are present in the transgenic animals as compared to wild-type controls. Increasing the expression of the stretch-activated cation channel in osteoblasts increases bone mineral density without the induction of abnormal bone development.

Thus, the present invention is directed to a non-human transgenic animal, particularly a murine, more particularly a mouse, which contains a nucleotide construct capable of altered expression of α-ENaC.

The present invention is also directed to an isolated and purified nucleotide construct which includes one of the following nucleotide sequences:

a) the nucleotide sequence of SEQ ID NO: 1, or its complement, or any portion of the nucleotide sequence or complement which is at least 36 nucleotide residues in length;

b) a nucleotide sequence which has at least 80% homology, preferably 95% homology, and most preferably, 98% homology with SEQ ID NO: 1; and

c) any portion of the nucleotide sequence of (b) which is at least 36 nucleotide residues in length.

Preferably, said sequence encodes a protein having α-ENaC activity.

In another aspect, the present invention is directed to a transgenic animal, particularly a murine, more particularly a mouse, which has at least one cell which contains a recombinant DNA sequence which includes one of the following nucleotide sequences:

a) the nucleotide sequence of SEQ ID NO: 1, or its complement, or any portion of the nucleotide sequence or complement which is at least 36 nucleotide residues in length;

b) a nucleotide sequence which has at least 80% homology, preferably at least 95% homology, and most preferably, at least 98% homology with SEQ ID NO: 1; and

c) any portion of the nucleotide sequence of (b) which is at least 36 nucleotide residues in length.

Preferably, said sequence encodes a portion having α-ENaC activity.

In still another aspect, the invention is directed to a method of producing a non-human mammal with altered expression of α-rENaC in osteoblasts which includes:

a) providing a vector construct containing α-rENaC; and

b) incorporating the vector construct into the genome of the non-human mammal such that the non-human mammal has altered expression of α-rENaC in osteoblasts.

The invention is also directed to a method of producing a transgenic non-human mammal with altered expression of stretch-activated cation channel in cells responsive to osteocalcin. The method comprises the following steps:

a) introducing a transgene into an osteocyte cell of the mammal, the transgene comprising a vector nucleotide sequence pKBpA wherein the stretch-activated cation channel (α-rENaC) is inserted into the pKBpA such that the stretch-activated cation channel gene is expressed; and

b) identifying a mammal which contains the stably integrated transgene and has the ability to express the stretch-activated cation channel transgene in osteoblasts.

The present invention is further directed to a method of producing a progeny of non-human mammal heterozygous for the transgene. The method comprises the following steps:

a) mating a first non-human mammal with a second non-human mammal, wherein the first non-human mammal expresses altered levels of stretch-activated cation channel in osteoblasts, and wherein the second non-human mammal expresses normal levels of stretch-activated cation channel in osteoblasts; and

b) selecting progeny derived from mating of step a) which are heterozygous for the transgene.

The present invention is further directed to a method of producing a progeny of non-human mammal homozygous for the transgene. The method comprises the following steps:

a) mating a first non-human mammal with a second non-human mammal, wherein the first non-human mammal and the second non-human mammal express altered levels of stretch-activated cation channel in osteoblasts; and

b) selecting progeny derived from mating of step a) which are homozygous for the transgene.

Other objects and features will be in part apparent and in part pointed out hereinafter.

### DETAILED DESCRIPTION OF THE INVENTION

The contents of each of the references cited herein are herein incorporated by reference.

The stretch-activated cation channel which is a macromolecule of the alpha-subunit of the epithelial sodium channel is expressed at high levels in the osteoblast lineage, especially osteocytes, and may be the mechanism by which cells sense fluid flow or mechanical strain. As a result of ion conductance through the stretch-activated cation channel, osteoblasts express a depolarization of the cell membrane potential, an increase in cytosolic calcium, cell volume regulation, and activation of cell anabolism. Increased anabolic activity causes stimulation of bone formation resulting in an accretion of bone and an increase in skeletal/mineral density and strength. As a result, pharmacological agents which can activate the stretch-activated cation channel will serve to stimulate bone formation. Cellular specificity is provided by the sensitivity of the osteocyte and osteoblast to mechanical strain resulting in increased insertion of the stretch-activated cation channel into the plasma membrane. Other cells in the body do not respond to the same forces by stretch-activated cation channel insertion. This is a unique mechanism of regulation and provides cell specificity for targets of the stretch-activated cation channel. Thus, other cells without stretch-activated cation channel on the membrane will not be sensitive to agents that recognize and activate the stretch-activated cation channel.

To better understand the physiologic functions of the stretch-activated cation channel, the present invention provides compositions, constructs, cells and animals with altered stretch-activated cation channel expression. In particular, novel mice that possess the capability to express altered levels of stretch-activated cation channel in osteoblasts are provided.

These genetically altered mice are important for the investigation of the role of the stretch-activated cation channel in bone in vivo, and for the development of pharmacological agents which activate the stretch-activated cation channel. Such mouse models are also important to establish the physiological function of the stretch-activated cation channel, both acting alone and in combination with one or more other proteins. These mice may also serve as a source of cells for culture for the above purposes.

The term “transgenic” refers to an organism which contains stably inherited genetic material which was inserted into the organism or its ancestors by molecular genetic manipulation.

The term “transgene” refers to the genetic material which was inserted.

The term “genetically” refers to a trait which is conferred by the translation of genetic material in an animal.

The term “recombinant” refers to any new cell, individual, or nucleic acid molecule, the nucleotide sequence of which is altered due to laboratory manipulation, or the natural process of in vivo recombination between a laboratory-manipulated sequence and a heterologous genome sequence.

The term “descendants” and “progeny” refer to any and all future generations derived or descending from a particular mammal, whether the mammal is heterozygous or homozygous for the nucleotide construct.

The term “% homology” refers to the degree of similarity between two nucleic acid sequences. The % homology may be determined by calculating the percentage of the nucleotides in the candidate sequence that are the same as the known nucleic acid sequence, after aligning the sequence and introducing gaps, if necessary, to achieve the maximum percent homology, and not considering any conservative substitutions as part of the sequence identity.

The procedures disclosed herein which involve the molecular manipulation of nucleic acids are known to those skilled in the art. See generally Fredrick M. Ausubel et al. (1995), “Short Protocols in Molecular Biology,” John Wiley and Sons, and Joseph Sambrook et al. (1989), “Molecular Cloning, A Laboratory Manual,” second ed., Cold Spring Harbor Laboratory Press, which are both incorporated herein by reference.

Generation of pKBpA/α-rENaC Positive Non-Human Mammals

The present invention provides non-human mammals which express stretch-activated cation channel transgene in osteoblasts. Present in these mice are osteoblast cells which resemble those found in wild-type animals. Such pKBpA/α-rENaC animals are generated by transforming the mammals with a transgene comprising a α-rENaC cDNA or its variant inserted into a pKBpA gene and fused to a promoter such as an osteocalcin promoter. Since osteoblast cell activity and structure is very similar among mammals, any non-human mammal which is amenable to transformation technology is a suitable subject for this procedure.

To perform targeted mutatgenesis of α-rENaC, an α-rENaC construct capable of altered expression of α-rENaC is created by methods known in the art. A preferred construct comprises α-rENaC gene and flanking sequence, where a portion of the gene is replaced with a selectable marker, preferably lac-Z, and driven by the osteocalcin promoter. The rat stretch-activated cation channel (α-rENaC) was cloned using methods known in the art. Human α-ENaC (α-hENaC) has similar homology with rat α-ENaC (α-rENaC) and may be cloned and used as an alternative to α-rENaC. See generally, Neil Kizer et al., (1997) Proc. Natl. Acad. Sci. USA, 94, 1013-18, Chun Cheng et al., (1998) J. Biol. Chem. 273, 22693-700, which are both incorporated herein by reference. To make the construct, the vector pKBpA is digested with suitable restriction enzymes, and α-rENaC, the promoter and the marker are inserted using ligation enzymes by methods known in the art.

The lac-Z marker may be used for incorporation because it provides a readily detectable signal of successful construct incorporation based on its activity producing a blue color. However, any marker nucleotide sequence (with or without its own promoter sequence) may be utilized which provides a detectable signal, such as of antibiotic resistance or the expression of a measurable quantity of a particular phenotypic trait. A positive/negative combination of markers may be used.

The targeting construct is used to transform mice, for example by electroporating mouse embryonic stem (ES) cells in the presence of the construct. Insertion of the nucleotide construct into the ES cells can also be accomplished by other means such as by microinjection. The ES cell clones carrying this mutation obtained from homologous recombination in two separate transfections are preferably injected into blastocytes to generate highly chimeric mice. The blastocyst may be obtained by perfusing the uterus of a pregnant female, and preferably are selected at a stage of development where the injected ES mutant cells readily will be incorporated (about 3-4 days in mice). Also, the blastocytes may have an identifiable phenotype difference from that of the incorporated ES cells (e.g., coat color) so that the chimeric mice are easily selected.

The blastocytes with incorporated ES mutants are then implanted into the uterus of a pseudopregnant mouse leading to the birth and selection of the chimeric mice. The α-rENaC transgene is transmitted into the germline of the chimeric mice provided that the embryonic stem cells are maintained in an undifferentiated state and capable of being a stem cell for the transmission of the α-rENaC mutation to all mouse cells. Chimeric mice thus produced are preferably bred with mice to generate mice heterozygous for the α-rENaC mutation. These heterozygotes are then interbred to produce mice homozygous for α-rENaC.

These mice are useful as models for the determination of the role of α-rENaC in osteoblast formation as well as for the investigation of therapeutical regimens which will stimulate bone formation in humans and other mammals. These mice may be used to screen for drugs for the development of such therapies. Screening for useful drugs generally involves administering the prospective drug at various dosages and assessing its impact on a physiological response of interest. These transgenic mice could be used to assess such drug candidates to determine whether activation of the desired physiological response is achieved.

The following examples illustrate the invention, but are not to be taken as limiting the various aspects of the invention so illustrated.

### EXAMPLES

Osteoblasts respond to chronic mechanical stimulation by increasing matrix protein synthesis, increasing the activity of non-selective, stretch-activated cation channels resident in the plasma membrane. Applicants created a vector construct (pKBpA/α-rENaC) containing stretch-activated cation channel (α-rENaC) in which transcription was driven by the osteocalcin promoter. This vector was used to generate a transgenic mouse strain which alters the expression of stretch-activated cation channel in cells responsive to osteocalcin. Animals heterozygous and homozygous for the transgene were generated through selective breeding and were screened for genotype by Southern blot. Northern blot analysis on freshly isolated osteoblasts indicate that cells from pKBpA/α-rENaC positive mice contains 46%+/−15% more α-rENaC mRNA than do wild-type litter mates. Additionally, pKBpA/α-rENaC positive mice produce significantly larger transients as measured by the highest level of [Ca⁺⁺]ᵢ observed after membrane strain application, 344+/−45 nM as compared to 120+/−19 nM for transgene negative controls. Furthermore, swelling-induced calcium entry into pKBpA/a-rENaC positive cells appears to have occurred at a rate 15 times greater than in control cells based on the initial rate of increase observed after swelling (7.9 nM/sec and 0.51 nM/sec). Measures of bone mineral density of femurs removed from 3 month old transgenic mice indicate a 15% increase in ashed density from 1.54 g/cc to 1.78 g/cc. This increase in bone mineral density was achieved while ashed weight normalized to bone dry weight remained the same ±2%, a further indication that normal bone phenotype was maintained.

As shown above, mice that have altered expression of the α-rENaC gene allow researchers to perform controlled experiments in mammals. That is, one can directly determine the role of α-rENaC in experimental models by comparing bone formation of pKBpA/α-rENaC positive mice versus wild-type mice. In the examples above, no somatic abnormalities were observed in the transgenic mice compared to wild type litter mates. Histomorphometric analysis of femurs indicated no apparent changes in the growth plate, cortical or trabecular bone. Length and width measurements of femur, tibia, vertebra and skull were found to be statistically the same for transgenic and wild type animals. These data indicate that by increasing expression of the stretch-activated cation channel, bone mineral density can be manipulated without the induction of abnormal bone growth.

In view of the above, it will be seen that the several objects of the invention are achieved.

As various changes could be made in the above compositions and processes without departing from the scope of the invention, it is intended that all matter contained in the above description be interpreted as illustrative and not in a limiting sense.

What is claimed is:

1. A transgenic non-human mammal comprising a nucleotide construct capable of enhanced expression of stretch-activated cation channel in osteoblasts relative to wild-type littermate.

2. The mammal of claim 1 wherein the mammal is a murine.

3. A transgenic non-human mammal which has at least one osteoblast cell which contains a recombinant DNA sequence which includes one of the following nucleotide sequences:
a) the nucleotide sequence of SEQ ID NO: 1, or its complement, or any contiguous portion of the nucleotide sequence or complement which is at least 36 nucleotide residues in length;
b) a nucleotide sequence which has at least 80% homology with SEQ ID NO: 1; and
c) any contiguous portion of the nucleotide sequence of (b) which is at least 36 nucleotide residues in length, and which at least one osteoblast cell is capable of enhanced expression of stretch-activated cation channel relative to such cell without the recombinant DNA sequence.

4. The mammal of claim 3 wherein the mammal is a murine.

5. A method of producing a non-human mammal with enhanced expression of stretch-activated cation channel in osteoblasts relative to the wild-type littermate comprising:
a) providing a vector construct containing a transgene encoding a protein having stretch-activated cation channel activity; and
b) incorporating the vector construct into the genome of the non-human mammal such that the non-human mammal has enhanced expression of stretch-activated cation channel in osteoblasts.

6. A method of producing progeny of a non-human mammal heterozygous for a stretch-activated cation channel transgene comprising:
a) mating a first non-human mammal with a second non-human mammal, wherein the first non-human mammal expresses enhanced levels of stretch-activated cation channel in osteoblasts relative to a wild-type litter mate, and wherein the second non-human mammal expresses normal levels of stretch-activated cation channel in osteoblasts; and
b) selecting progeny obtained from said mating of step a) which are heterozygous for the transgene.

7. A method of producing a progeny of a non-human mammal homozygous for a stretch-activated cation channel transgene comprising:
a) mating a first non-human mammal with a second non-human mammal, wherein the first non-human mammal and the second non-human mammal express enhanced levels of stretch-activated cation channel in osteoblasts relative to a wild-type litter mate; and
b) selecting progeny obtained from said mating of step a) which are homozygous for the transgene.

8. A transgenic non-human mammal as set forth in claim 3 wherein the at least one osteoblast cell is capable of producing at least 31% more stretch-activated cation channel mRNA than such cell without the recombinant DNA sequence.

9. A transgenic murine comprising a nucleotide construct capable of enhanced expression of stretch-activated cation channel in osteoblasts relative to a wild-type littermate, wherein the nucleotide construct comprises a gene encoding a stretch-activated cation channel operably linked to an osteocalcin promoter.

10. A transgenic murine as set forth in claim 9 wherein the osteoblast cells comprising the nucleotide construct are capable of producing at least 31% more stretch-activated cation channel mRNA than such cells without the nucleotide construct.
